(19) United States
(12) Patent Application Publication
(10) Pub. No.: US 2005/0175717 A1
(43) Pub. Date: Aug. 11, 2005

(54) (Cosmetic) composition, process of making, uses thereof

(75) Inventors: Roland De La Mettrie, Le Vesinet (FR); Rainer Muller, Leopoldshafen (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/041,855

(22) Filed: Jan. 25, 2005

Related U.S. Application Data
(60) Provisional application No. 60/545,187, filed on Feb. 18, 2004.

(30) Foreign Application Priority Data
Jan. 29, 2004 (FR) 0400847

(51) Int. Cl. A61K 35/78

(52) U.S. Cl. 424/725; 424/745; 424/764; 424/750; 424/770; 424/771; 424/767; 424/768; 424/757

(57) ABSTRACT

The invention relates to a process for preparing a composition for the cosmetic treatment of keratin materials by percolating fluid comprising at least steam, at a pressure of at least 3 bar, through at least one non-colouring plant substance in solid or pasty form. The invention also relates to the composition made, and to a process for treating keratin materials using the composition.

### REFERENCE TO PRIOR APPLICATIONS

This application claims priority to U.S. provisional application 60/545,187 filed Feb. 18, 2004, and to French patent application 0400847 filed Jan. 29, 2004, both incorporated herein by reference.

### FIELD OF THE INVENTION

The present invention relates to a process for preparing a composition useful especially for the cosmetic treatment of keratin materials such as the skin and keratin fibres, for example the hair, and also to a cosmetic process for treating keratin materials using this composition.

More particularly, the invention relates to a process for preparing a composition comprising a step of percolating fluid comprising at least steam, at a pressure of at least 3 bar, through at least one non-colouring plant substance in solid or pasty form, preferably chosen from plant substances with softening properties, anti-inflammatory properties, antiseptic properties, antiperspirant properties, calmative properties, cicatrizing properties, tonifying properties, properties promoting containment of the blood and lymphatic microcirculation, texturing properties, antioxidant properties, foaming or emulsifying properties, photoprotective properties, thickening, absorbing properties and/or odorizing properties. The invention further includes the compositions prepared, and uses thereof.

### BACKGROUND OF THE INVENTION

In cosmetics, it is always sought to improve the cosmetic properties of keratin materials. It is thus known practice to use in topical-application compositions treating agents derived from plants, for combating external attacking factors such as pollution and ultraviolet radiation, for combating damage to keratin fibres or the skin, especially the signs of ageing of the skin, for moisturizing and nourishing keratin fibres and the skin, and for giving the skin and the other keratin materials treated with these compositions all the benefits associated with these treating agents.

In particular, these treating agents may be used for treating the skin, the hair, the eyelashes, the lips and the nails, for example for nourishing or moisturizing the skin, protecting it against external agents, removing dead skin, decreasing wrinkles and fine lines and also coloured marks (pigmentation defect or marks appearing with age, in particular on the hands, the neck or the face).

The use of treating agents derived from plants is known in cosmetics, especially for obtaining softening, anti-inflammatory, anti-pruriginous, antiseptic, antiperspirant, astringent, calmative, cicatrizing, firming, tonifying, fragrancing, foaming, odorizing, absorbent, thickening, antioxidant, emulsifying, photoprotective and/or texturing properties. The formulations containing such active substances are generally obtained via various processes such as maceration, digestion, decoction, infusion or lixifiation.

However, (cosmetic treatment) compositions containing such treatment agents are generally aqueous compositions in which the agents must be dissolved. The lack of solubility of these compounds reduces the treating nature of these compositions. In addition, this solubility criterion reduces the number of treatment agents that originate from plants and can be used for the cosmetic treatment of keratin materials, as well as the number of compositions containing them that can be offered to consumers.

Moreover, numerous natural compounds have the drawback of being unstable with respect to water or other organic or inorganic compounds currently used in cosmetics, namely they are subject to degradation by a hydrolysis mechanism or reaction with the organic or inorganic compounds, which leads to (cosmetic) compositions that are ineffective and/or whose appearance, smell and/or touch are unacceptable for the consumer, within a few days.

### DETAILED DESCRIPTION OF THE PREFERRED EMBODIMENTS

It has now been discovered, surprisingly, that by using a novel process for preparing a (cosmetic) composition useful for example for treating keratin materials, compositions that are more or less concentrated in treatment agent(s) originating from plants may be obtained in a very short time, for example of less than 2 minutes, according to need, and especially without preserving agent, enabling the solubility and stability problems outlined above to be overcome.

This process may be performed simply and is suited to the consumer. A pressurized fluid, the temperature of which is preferably greater than or equal to 30° C., is passed for a short time through at least one non-colouring plant substance in solid or pasty form, preferably solid and even more preferably pulverulent.

The invention process enables the use of fresh or dried non-colouring plant substances, in order to obtain aqueous compositions that are concentrated in treatment agents originating from these non-colouring plant substances, and thus to improve their effectiveness while at the same time avoiding the degradation of the treatment agents.

The compositions prepared according to this process may have limited stability on storage, which is not a drawback in this case since the process leads to a ready-to-use composition intended to be used quickly after its preparation, for example within five minutes of preparation, especially after cooling to a temperature that is acceptable for keratin materials, preferably below 60° C., better still below 50° C. The composition may also be used up to one week or more after its preparation, depending on the rate of degradation of the treatment agent.

Given the very short preparation time, the (cosmetic treatment) compositions may be prepared “on demand” by mixing the active compounds, i.e. according to the desired cosmetic properties, in a particular embodiment.

According to another embodiment, since the active compounds may be packaged in a ready-to-use device, it is not necessary to determine beforehand the concentrations of active compounds, which limits the measuring errors by the user.

In addition, the process according to the invention makes it possible to avoid the use of multi-compartment bottles, which makes the process particularly economical and safer for the user.

The composition thus obtained may be used alone or as a mixture with another composition.

One subject of the invention is thus a process for preparing a (cosmetic) composition for treating keratin materials, comprising a step of percolating a fluid at a pressure of at least 3 bar through at least one non-colouring plant substance in solid or pasty form.

Another subject of the invention is a composition obtained via the process according to the invention.

A subject of the invention is also the use of the composition obtained according to the process of the invention, for the cosmetic treatment of keratin materials such as the skin, the lips, the hair, the eyelashes and the nails.

Another subject of the invention is a process for the cosmetic treatment of keratin materials of human beings, comprising a) the preparation of a ready-to-use (cosmetic) composition by percolating a fluid at a pressure of at least 3 bar through at least one non-colouring plant substance in solid or pasty form, and b) the application of a composition obtained in step a) to the keratin materials.

The application to the keratin materials may, depending on the nature of the non-colouring plant substances, be carried out by the topical route or the oral route, and preferably by the topical route.

Another subject of the invention is a packaging device for performing the preparation process of the present invention.

Other subjects, characteristics, aspects and advantages of the invention will emerge even more clearly on reading the further nonlimiting description and the example that follows.

According to the invention, the process for preparing a (cosmetic) composition for treating keratin materials comprises a step of percolating a fluid, at a temperature preferably of greater than or equal to 30° C., better still ranging from 30° C. to 150° C., and even more preferably from 40° C. to 120° C., at a pressure of at least 3 bar (3×105 Pa), through at least one non-colouring plant substance in solid or pasty form.

Percolation is a movement of fluid through a porous medium, allowing the passage of the fluid due to the action or effect of pressure.

The fluid preferably comprises at least steam. It may also be accompanied by liquid water, and/or by one or more cosmetically acceptable liquid and/or gaseous solvents. Preferably, the fluid is steam accompanied by liquid water.

Examples of useful organic solvents that may be mentioned include C1-C4 lower alcohols, such as ethanol and isopropanol; polyols and polyol ethers, for instance 2-butoxyethanol, propylene glycol, propylene glycol monomethyl ether, diethylene glycol monomethyl ether and diethylene glycol monoethyl ether, and also aromatic alcohols, for instance benzyl alcohol or phenoxyethanol, and mixtures thereof.

The non-colouring plant substance is in solid form or in pasty form, preferably in solid form and even more preferably pulverulent.

The non-colouring plant substance may be fresh or dried and be in any form including in the form of leaves, roots, stems, bark, flowers, petals, pistils, fruits or seeds, or extracts obtained from the whole plant or its different parts.

For the purposes of the present invention, the term “pasty form” means a consistency intermediate between a solid phase and a liquid phase. The viscosity of this pasty phase is preferably greater than 0.1 Pa.s and even more preferably greater than 1 Pa.s, at 25° C. with a shear rate of 10 s-1.

The term “keratin materials” means the skin, the eyelids, the scalp, the lips, and/or the integuments such as the nails, and keratin fibres such as the eyelashes, the eyebrows and the hair.

The process of the present invention may be performed using a standard device for generating a pressurized fluid, at a temperature preferably of greater than or equal to 30° C., better still ranging from 30° C. to 150° C., and even more preferably ranging from 40° C. to 120° C. Such a device comprises a pressure-resistant chamber, equipped with a thermal block, and also a circuit for conveying the fluid produced to the non-colouring plant substance.

According to another embodiment, the device additionally comprises a reservoir of liquid(s) and also a pump for conveying the liquid(s) to the chamber.

The liquid contained in the reservoir is preferably water, or a mixture of water and of one or more cosmetically acceptable solvents. Preferably, the liquid is water.

A device that is particularly useful for performing the process of the present invention is a coffee machine of the “espresso” type. Such machines are well known in the art. For example, these machines are described in patents AT 168 405, U.S. Pat. No. 2,688,911, DE 324 33 870 and IT 1 265 636.

According to one particular embodiment of the invention, the percolation step is performed with a fluid at a temperature of greater than 30° C., preferably ranging from 30° C. to 150° C., and even more preferably from 40° C. to 120° C., under a pressure ranging from 3 to 30 bar (3×105 to 3×106 Pa), preferably of at least 4 bar (4×105 Pa), more preferably greater than or equal to 10 bar (106 Pa), and most particularly ranging from 10 to 30 bar (106 to 3×106 Pa).

A (cosmetic) composition containing at least one non-colouring plant substance in solid or pasty form may be used directly in the device for generating the pressurized fluid in a container intended for this use. It may also be packaged in a particular packaging device, of single-dose type, comprising a closed housing delimited by at least one wall that is at least partially permeable to fluid comprising at least steam, under a pressure of at least 3 bar. Such devices are described, for example, in patent applications WO 00/56629, EP 512 470, WO 99/03573 or U.S. Pat. No. 5,897,899. These packaging devices are generally airproof, humidityproof and/or lightproof.

According to one particular embodiment, the housing is delimited by two sealed sheets. According to another embodiment, the housing is delimited by a tray closed with a lid.

These devices may be manufactured from woven or nonwoven, plastic or plant materials, for example cellulose, metal such as aluminium, or composite materials. Such devices are described, for example, in patent applications WO 00/56629, EP 512 470, U.S. Pat. No. 5,897,899 or WO 99/03573.

The non-colouring plant substances that may be used in the process of the invention include those cited, for example, in the “Encyclopedia of common natural ingredients used in food, drugs and cosmetics” 2nd edition, Albert Y. Leung and Steven Foster, Wiley-Interscience, 1996.

They are preferably chosen from plant substances with softening properties, anti-inflammatory properties, antiseptic properties, antiperspirant properties, calmative properties, cicatrizing properties, tonifying properties, properties promoting containment of the blood and lymphatic microcirculation, texturing properties, antioxidant properties, foaming or emulsifying properties, photoprotective properties, thickening, absorbing properties and/or odorizing properties.

As examples of non-colouring plant substances as a function of their various properties, mention may especially be made of:
for their softening properties:
apricot, cornflower, great mullein, roman camomile, mayweed, field poppy, fenugreek, althaea, flax, lily, mallow, calendula, elder, linden, coltsfoot, psyllium, plantain, quince, peach, orange, cactus, apple;
for their anti-inflammatory properties:
   agrimony, hawthorn, heather, couch grass, juniper, althaea, elder, linden, fenugreek, gentian, lettuce, wild pansy, plantain, blackberry, rosemary, sage, black bryony, coltsfoot, everlasting, daisy;
for their antiseptic properties:
   garlic, agrimony, bilberry, burdock, oak, comfrey, eucalyptus, juniper, rose geranium, laurel, lavender, marjoram, mint, pine, rosemary, sandalwood, mother-of-thyme, thyme, sage, honeysuckle, everlasting, daisy, tansy;
for their antiperspirant properties:
   sage, oak, walnut, pine, horsetail, coltsfoot;
for their astringent properties:
   acacia, common yarrow, agrimony, lady's-mantle, madrone, sagebrush, comfrey, cypress, oak, dog rose, witch hazel, black mulberry, bilberry, hazel, walnut, nettle, poplar, plantain, blackberry, ratanhia, rose, purple lythrum, willow, tormentilla cinquefoil, red vine, sweet clover;
for their calmative properties:
   carrot, wild pansy, elder, linden, passionflower, basil, camphor, pear, apple, vine, lettuce, rose, ginger;
for their cicatrizing properties:
   St.-John's-wort, silverweed cinquefoil, calendula, mayweed, roman camomile, comfrey, common yarrow, common wormwood, agrimony, sagebrush, arnica, chervil, myrtle, periwinkle, plantain, poplar, primrose, sage, ragwort, elder, vervain, angelica, Dutchman's-pipe, alder, old-man wormwood, European bistort, birch, blessed thistle, juniper, medlar, eucalyptus, woundwort, avens, centaurea, cabbage, houseleek, strawberry, horsetail, European meadowsweet, coltsfoot, wild pansy, burdock, daisy, lily;
for their tonifying properties:
   St.-John's-wort, dog rose, mistletoe, Paraguay tea, senna, common wormwood, arnica, calamint, cinnamon, geranium, hyssop, marjoram, balm, parsley, Scotch pine, rosemary, savory, mother-of-thyme, basil, dog rose, gentian, hop, white laurel, mint, sage, tormentilla cinquefoil, common yarrow, agrimony, avens, European bistort, oak, quince, cypress, common horse chestnut, medlar, walnut, nettle, plantain, marsh-pepper smartweed, creeping cinquefoil, knotweed, purple lythrum, veronica, angelica, sagebrush, woodruff, juniper, mustard, cinchona, fumitory, nasturtium, cress, rockweed, butcher's-broom, tansy;
for their properties promoting containment of the blood and capillary microcirculation:
   blackcurrant, bilberry and grapeseed;
for their texturing properties:
   wheat, fucus;
for their antioxidant properties:
   rice, rosemary, sage, thyme, green tea, liquorice;
for their foaming or emulsifying properties:
   soapwort, ivy, butcher's-broom, quillaja bark, quillaja, greenbrier, quinoa, soya;
for their photoprotective properties:
   aloe, sunflower, liquorice, magnolia, resurrection lily;
for their thickening or absorbing properties:
   pea, wheat, potato, corn; and
for their odorizing properties:
   rosemary, violet, lavender and rose.

Preferably, the plant substances with softening properties are chosen from cornflower, field poppy, fenugreek, althaea, flax, mallow, elder and cactus; the plant substances with anti-inflammatory properties are chosen from couch grass, althaea, juniper, lettuce, sage, elder, rosemary and everlasting; the plant substances with antiseptic properties are chosen from burdock, comfrey, agrimony, juniper, rosemary and oak; the plant substances with antiperspirant properties are chosen from sage, pine, horsetail and oak; the plant substances with astringent properties are chosen from acacia, witch hazel, tormentilla cinquefoil, dog rose, ratanhia and comfrey; the plant substances with calmative properties are chosen from rose, passionflower, carrot, wild pansy and ginger; the plant substances with cicatrizing properties are chosen from arnica, European meadowsweet, sage, comfrey, elder, horsetail, European bistort, mayweed and lily; the plant substances with tonifying properties are chosen from mistletoe, Paraguay tea, senna, knotweed, sage, European bistort, common horse chestnut, rosemary and angelica; the plant substance with texturing properties is wheat; the plant substances with antioxidant properties are chosen from rosemary and thyme; the plant substances with foaming or emulsifying properties are chosen from soapwort, quillaja bark and greenbrier; the plant substance with photoprotective properties is aloe; the plant substances with thickening or absorbing properties are chosen from potato and corn; and the plant substances with odorizing properties are chosen from rosemary, lavender and rose.

The non-colouring plant substance(s) according to the invention may be used in the process of the invention as a mixture with one or more solid or pasty, and preferably pulverulent, adjuvants. The adjuvants may be chosen from clays, salts, anionic, nonionic, cationic or zwitterionic surfactants, natural or synthetic thickeners, optionally modified starch, glass beads, silica, Nylon, alumina, titanium dioxide, zeolites, polymethyl methacrylate (PMMA), chitosan, maltodextrin, cyclodextrin, mono- or disaccharides, for instance glucose, sucrose, sorbitol or fructose, zinc oxide, zirconium oxide, silicabades, talc, polyaspartic acid, borosilicates, especially calcium borosilicate, polyethylene, cotton, polytetrafluoroethylene (PTFE), cellulose and its derivatives, superabsorbent compounds, magnesium carbonate, calcium carbonate, corn seeds, polyacrylamide, porous hydroxyapatite, silk, collagen, sawdust, wrack powder, meals or extracts of wheat, rice, pea, lupin, soybean or barley, crosslinked polyvinylpyrrolidone, calcium alginate, active charcoal, and poly(vinylidene chloride/acrylonitrile) particles, especially those sold under the general name “Expancel®” by the company Akzo Nobel under the particular reference “Expancel® WE” or “Expancel DE”, and mixtures thereof.

When one or more adjuvants are present, the non-colouring plant substance(s) used in the invention is (are) preferably present in an amount ranging from 0.5% to 99% by weight, better still from 1% to 80% by weight and even more preferably from 2% to 60% by weight relative to the total weight of non-colouring plant substance(s) and adjuvant(s) in solid or pasty form.

Plants or plant extracts used in the process of the present invention, may be pretreated before the percolation step. The pretreatment can be drying, roasting, cryogrinding or freeze-drying.

The (cosmetic) composition for treating keratin materials obtained according to the process of the invention may contain, besides the treatment agent(s) and the component(s) of the fluid, i.e. water and/or cosmetically acceptable solvent(s), optionally all or some of the adjuvant(s) present in the mixture in solid or pasty form.

The invention also relates to a composition obtainable via the process according to the invention, the particularly preferred composition being free of preserving agents.

Using the preparation process of the invention, a (cosmetic treatment) composition is obtained, which may be applied directly to keratin materials, or which may be mixed with a cosmetically acceptable medium, or alternatively at least one additive conventionally used in cosmetics may be added thereto by an operator. At least two compositions obtained via the process of the invention may also be mixed together. The (cosmetic treatment) composition optionally resulting from the mixture(s) and/or addition(s) indicated above will be referred to hereinbelow as the final (cosmetic treatment) composition or final composition.

One particular embodiment of the invention consists in applying the composition obtained by means of a device not requiring any human intervention, and optionally equipped with a cooling means.

Another particular embodiment consists in ingesting the (cosmetic treatment) composition obtained according to the process of the invention, when no toxicity problem is known in the technique.

The amount of the non-colouring plant substance extract present in the final (cosmetic treatment) composition is not limited, and may generally be included in the interval ranging from 0.001% to 50% by weight approximately, preferably from 0.005% to 30% by weight, and even more preferably from 0.01% to 20% by weight, relative to the total weight of the final (cosmetic treatment) composition.

In the case where the (cosmetic) composition obtained via the process of the present invention is mixed with a cosmetically acceptable medium, such a medium generally comprises water or a mixture of water and of at least one organic solvent to dissolve the compounds that would not be sufficiently soluble in water.

The term “cosmetically acceptable” means a medium that is compatible with keratin materials and especially the skin, the lips and/or the integuments, and which additionally has an appearance, a feel, a smell and optionally a taste that is pleasant for the user.

Examples of organic solvents that may be mentioned include C1-C4 lower alcohols, such as ethanol and isopropanol; polyols and polyol ethers, for instance 2-butoxyethanol, propylene glycol, propylene glycol monomethyl ether and diethylene glycol monomethyl ether and diethylene glycol monoethyl ether, and also aromatic alcohols, for instance benzyl alcohol or phenoxyethanol, and mixtures thereof.

The solvents are preferably present in proportions preferably ranging from 1% to 40% by weight, and even more preferably from 5% to 30% by weight relative to the total weight of the final (cosmetic treatment) composition.

At least one additive conventionally used in cosmetics may also be added to the (cosmetic treatment) compositions obtained according to the process of the present invention. Examples of such additives that may be mentioned include anionic, cationic, nonionic, amphoteric or zwitterionic surfactants, or mixtures thereof; anionic, cationic, nonionic, amphoteric or zwitterionic polymers, or mixtures thereof; mineral or organic thickeners, and in particular anionic, cationic, nonionic and amphoteric polymeric associative thickeners; antioxidants; penetrating agents; sequestering agents; fragrances; buffers; dispersants; conditioning agents; film-forming agents; ceramides; preserving agents; opacifiers; and coloured or nacreous pigments.

The above additives are generally present in an amount for each of them of between 0.01% and 20% by weight relative to the weight of the final composition.

Needless to say, a person skilled in the art will take care to select this or these optional compounds such that the advantageous properties intrinsically associated with the (cosmetic) composition in accordance with the invention are not, or are not substantially, adversely affected by the addition(s) envisaged.

The pH of the final composition is preferably generally between 3 and 12 and more preferably between 5 and 11. It may be adjusted to the desired value using acidifying or basifying agents usually used in cosmetics, or alternatively using standard buffer systems.

Among the acidifying agents that may be mentioned, for example, are mineral or organic acids such as hydrochloric acid, orthophosphoric acid, sulfuric acid, carboxylic acids such as acetic acid, tartaric acid, citric acid and lactic acid, and sulfonic acids.

Among the basifying salts that may be mentioned, for example, are ammonia, alkaline carbonates, alkanolamines such as mono-, di- and triethanolamine and derivatives thereof, sodium hydroxide, potassium hydroxide and the compounds of the following formula: 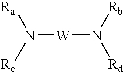
in which W is a propylene residue that is optionally substituted with a hydroxyl group or a C1-C4 alkyl radical; Ra, Rb, Rc and Rd, which may be identical or different, represent a hydrogen atom, a C1-C4 alkyl radical or a C1-C4 hydroxyalkyl radical.

The final (cosmetic treatment) composition may be in any of various forms, such as in the form of liquids, creams or gels, or in any other form that is suitable for treating and/or caring for keratin materials, and in particular keratin fibres, the skin or the scalp, or for making up the skin, the nails, the eyelashes or also the lips.

It can be used in particular for treating keratin materials such as the skin and keratin fibres, for example for toning the keratin fibres and the scalp.

The final (cosmetic treatment) composition may be used, for example, as a shampoo, a rinse-out or leave-in conditioner, a deep-down care mask, a shower gel, or a lotion or cream for treating keratin materials.

The present invention also relates to a cosmetic process for treating keratin materials, comprising the preparation of a (cosmetic treatment) composition according to the process as defined above, and its application to the keratin materials, for example by means of an operator or by means of a device not requiring any human intervention. The application time may range for example between 15 seconds and one hour.

Before application, the (cosmetic treatment) composition obtained according to the process of the invention may be mixed with a cosmetically acceptable medium and/or with one or more additives conventionally used in cosmetics, as described above.

Another embodiment includes preparing at least two (cosmetic treatment) compositions according to the process of the invention, mixing them together, and optionally adding a cosmetically acceptable medium and/or one or more additives conventionally used in cosmetics, as described above, and then in applying the final composition obtained to keratin materials.

The example below is intended to illustrate the present invention, but not limit it.

### EXAMPLE 1

The solid ingredients below are mixed together in the proportions indicated as weight percentages relative to the total weight of solid mixture:

| | |
|---|---|
| Total saponins of climbing ivy sold under the tradename | 50% |
| “Lierre saponines totales 50%” by the company Indena | |
| Magnesium sulphate | 50% |

5 g of this mixture are placed in a commercial expresso machine. Steam is then passed through until a composition (A) having a final volume of 50 ml is obtained.

A (cosmetic treatment) composition ready to be applied to the hair and the scalp is thus obtained. This composition has a toning effect on the fibre and the scalp.

Two parts by weight of composition (A) can be added to one part by weight of an aqueous composition (B) containing 1% by weight of hydroxyethylcellulose, to facilitate the application.

The above written description of the invention provides a manner and process of making and using it such that any person skilled in this art is enabled to make and use the same, this enablement being provided in particular for the subject matter of the appended claims, which make up a part of the original description and including a process for preparing a (cosmetic) composition useful for treating keratin materials, wherein it comprises a step of percolating a fluid under a pressure of at least 3 bars through at least one non-colouring plant substance in solid or pasty form, the at least one non-colouring plant substance being chosen from plant substances with at least one of softening properties, anti-inflammatory properties, antiseptic properties, antiperspirant properties, calmative properties, cicatrizing properties, tonifying properties, properties promoting containment of the blood and lymphatic microcirculation, texturing properties, antioxidant properties, foaming or emulsifying properties, photoprotective properties, thickening, absorbing properties and/or odorizing properties, the composition prepared, and a process using the composition.

As used herein, the phrases “selected from the group consisting of,” “chosen from,” “selected from,” and the like include mixtures of the specified materials.

Where compounds are described as, e.g, “cellulose and its derivatives” and the like an alternate is “cellulose and cellulose compounds” where cellulose derivatives and compounds share a common core/structure with cellulose. One of ordinary skill in the art knows how to identify derivatives and compounds of compound X based on the structure of X and the similarity in structure of the derivatives and compounds.

Where a term is enclosed by parentheses it is an optional modifier. For example, the term “(cosmetic treatment) composition” describes both a composition in general and a cosmetic treatment composition.

All references, patents, applications, tests, standards, documents, publications, brochures, texts, articles, etc. mentioned herein are incorporated herein by reference: Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A process for preparing a composition, comprising percolating fluid comprising at least steam, at a pressure of at least 3 bar, through at least one non-colouring plant substance in solid or pasty form, the at least one non-colouring plant substance being chosen from plant substances with at least one of softening properties, anti-inflammatory properties, antiseptic properties, antiperspirant properties, calmative properties, cicatrizing properties, tonifying properties, properties promoting containment of the blood and lymphatic microcirculation, texturing properties, antioxidant properties, foaming or emulsifying properties, photoprotective properties, thickening, absorbing properties and/or odorizing properties.

2. A process according to claim 1, wherein the fluid comprises steam and at least one of liquid water and one or more cosmetically acceptable liquid and/or gaseous solvents.

3. A process according to claim 1, comprising percolating fluid comprising at least steam, at a pressure of at least 3 bar, through at least one plant substance with softening properties chosen from cornflower, field poppy, fenugreek, althaea, flax, mallow, elder and cactus.

4. A process according to claim 1, comprising percolating fluid comprising at least steam, at a pressure of at least 3 bar, through at least one plant substance with anti-inflammatory properties chosen from couch grass, althaea, juniper, lettuce, sage, elder, rosemary and everlasting.

5. A process according to claim 1, comprising percolating fluid comprising at least steam, at a pressure of at least 3 bar, through at least one plant substance with antiseptic properties chosen from burdock, comfrey, agrimonyl, juniper, rosemary, and oak.

6. A process according to claim 1, comprising percolating fluid comprising at least steam, at a pressure of at least 3 bar, through at least one plant substance with antiperspirant properties chosen from sage, pine, horsetail and oak.

7. A process according to claim 1, comprising percolating fluid comprising at least steam, at a pressure of at least 3 bar, through at least one plant substance with astringent properties chosen from acacia, witch hazel, tormentilla cinquefoil, dog rose, ratanhia and comfrey.

8. A process according to claim 1, comprising percolating fluid comprising at least steam, at a pressure of at least 3 bar, through at least one plant substance with calmative properties chosen from rose, passionflower, carrot, wild pansy and ginger.

9. A process according to claim 1, comprising percolating fluid comprising at least steam, at a pressure of at least 3 bar, through at least one plant substance with cicatrizing properties chosen from arnica, European meadowsweet, sage, comfrey, elder, horsetail, European bistort, mayweed and lily.

10. A process according to claim 1, comprising percolating fluid comprising at least steam, at a pressure of at least 3 bar, through at least one plant substance with tonifying properties chosen from mistletoe, Paraguay tea, senna, knotweed, sage, European bistort, common horse chestnut, rosemary and angelica.

11. A process according to claim 1, comprising percolating fluid comprising at least steam, at a pressure of at least 3 bar, through at least one plant substance with properties promoting the containment of the blood and lymphatic microcirculation chosen from blackcurrant, bilberry and grapeseed.

12. A process according to claim 1, comprising percolating fluid comprising at least steam, at a pressure of at least 3 bar, through wheat.

13. A process according to claim 1, comprising percolating fluid comprising at least steam, at a pressure of at least 3 bar, through at least one plant substance with antioxidant properties chosen from rosemary and thyme.

14. A process according to claim 1, comprising percolating fluid comprising at least steam, at a pressure of at least 3 bar, through at least one plant substance with foaming or emulsifying properties chosen from soapwort, quillaja bark and greenbrier.

15. A process according to claim 1, comprising percolating fluid comprising at least steam, at a pressure of at least 3 bar, through aloe.

16. A process according to claim 1, comprising percolating fluid comprising at least steam, at a pressure of at least 3 bar, through at least one plant substance with thickening or absorbing properties chosen from potato and corn.

17. A process according to claim 1, comprising percolating fluid comprising at least steam, at a pressure of at least 3 bar, through at least one plant substance with odorizing properties chosen from rosemary, lavender and rose.

18. A process according to claim 1, wherein the non-colouring plant substance in solid or pasty form is present in a mixture with an adjuvant.

19. A process according to claim 18, wherein the adjuvant is chosen from clays, salts, anionic, nonionic, cationic or zwitterionic surfactants, natural or synthetic thickeners, optionally modified starch, glass beads, silica, Nylon, alumina, titanium dioxide, zeolites, polymethyl methacrylate (PMMA), chitosan, maltodextrin, cyclodextrin, mono- or disaccharides, zinc oxide, zirconium oxide, silicabades, talc, polyaspartic acid, borosilicates, polyethylene, cotton, polytetrafluoroethylene (PTFE), cellulose and its derivatives, superabsorbent compounds, magnesium carbonate, calcium carbonate, corn seeds, polyacrylamide, porous hydroxyapatite, silk, collagen, sawdust, wrack powder, meals or extracts of wheat, rice, pea, lupin, soybean or barley, crosslinked polyvinylpyrrolidone, calcium alginate, active charcoal, poly(vinylidene chloride/acrylonitrile) particles, and mixtures thereof.

20. A process according to claim 18, wherein the non-colouring plant substance is present in an amount ranging from 0.5% to 99% by weight relative to the total weight of non-colouring plant substance(s) and adjuvant in solid or pasty form.

21. A process according to claim 1, wherein the percolation step is performed with a fluid at a pressure ranging from 3 to 30 bar.

22. A process according to claim 21, wherein the percolation step is performed with a fluid at a pressure of at least 10 bar.

23. A process according to claim 1, wherein non-colouring plant substance are pretreated before the percolation step.

24. A process according to claim 23, wherein the pretreatment is at least one of drying, roasting, cryogrinding and freeze-drying.

25. A composition obtained by a process comprising percolating fluid comprising at least steam, at a pressure of at least 3 bar, through at least one non-colouring plant substance in solid or pasty form, the at least one non-colouring plant substance being chosen from plant substances with at least one of softening properties, anti-inflammatory properties, antiseptic properties, antiperspirant properties, calmative properties, cicatrizing properties, tonifying properties, properties promoting containment of the blood and lymphatic microcirculation, texturing properties, antioxidant properties, foaming or emulsifying properties, photoprotective properties, thickening, absorbing properties and/or odorizing properties.

26. The composition according to claim 25, wherein said composition comprises no preserving agent.

27. A process, comprising applying the composition of claim 25 to keratin material.

28. The process according to claim 27, comprising application to at least one of the lips, the hair, the eyelashes and the nails.

29. A device for packaging a composition, comprising a closed housing delimited by at least one wall that is at least partially permeable to fluid at a pressure of at least 3 bar, the device having therein a composition comprising at least one non-colouring plant substance chosen from plant substances with at least one of softening properties, anti-inflammatory properties, antiseptic properties, antiperspirant properties, calmative properties, cicatrizing properties, tonifying properties, properties promoting containment of the blood and lymphatic microcirculation, texturing properties, antioxidant properties, foaming or emulsifying properties, photoprotective properties, thickening, absorbing properties and/or odorizing properties, the device housing being optionally bounded by two sealed films or by a tray closed by a lid.
